(19) United States
(12) Patent Application Publication
(10) Pub. No.: US 2017/0333500 A1
(43) Pub. Date: Nov. 23, 2017

(54) Genetically Modified Bacteriophage (Bio-Phage)

(71) Applicant: Roc Hatfield, Safety Harbor, FL (US)

(72) Inventor: Roc Hatfield, Safety Harbor, FL (US)

(21) Appl. No.: 15/597,919

(22) Filed: May 17, 2017

Related U.S. Application Data
(60) Provisional application No. 62/337,370, filed on May 17, 2016.

(51) Int. Cl. A61K 35/76; C12N 7/00

(52) U.S. Cl. CPC A61K 35/76; C12N 7/00; C12N 2795/00021; C12N 2795/00051; C12N 2795/00032

(57) ABSTRACT

The present invention describes a genetically modified Staphylococcus aureus bacteriophage VDX-10 comprising the DNA of the bacteriophage VDX-10 being altered by inserting a gene sequence that increases the ability of the bacteriophage to replicate faster as compared to unmodified VDX-10 bacteriophage; a method of producing the genetically modified Staphylococcus aureus bacteriophage VDX-10; and a method of treating infection in a patient by administering an amount of the genetically modified Staphylococcus aureus bacteriophage effective to eliminating the Staphylococcus bacteria cells, where the infection can be Ventilator-Associated Pneumonia (VAP) or bacteremia as incited by methicillin-resistant Staphylococcal aureus (MRSA) or methicillin-sensitive Staphylococcal aureus (MSSA).

The current application claims a priority to a U. S. Provisional application of 62/337,370 filed May 17, 2016.

### FIELD OF THE INVENTION

The present invention relates generally to a method for producing a genetically modified Staphylococcus aureus bacteriophage and the use of this genetically modified bacteriophage in the treatment of infections caused by Staphylococcus bacteria. More specifically, the present invention relates to a genetically modified and enhanced Staphylococcus aureus bacteriophage VDX-10 and its use in the treatment Ventilator-Associated Pneumonia (VAP) or bacteremia as incited by Staphylococcus aureus.

### BACKGROUND OF THE INVENTION

Bacteriophages are ubiquitous viruses, found wherever bacteria exist. Bacteriophages (Phages) are viruses that infect, parasitize and kill specific bacteria (FIG. 1). Bacteriophages are composed of proteins that encapsulate a DNA or RNA and may have relatively simple or elaborate structures (FIG. 2). Their genomes may encode as few as four genes and as many as hundreds of genes. Bacteriophages replicate within the bacterium following the injection of their genome into cytoplasm. Phages reproduce by inserting their DNA into the DNA of a host bacterial cell. The phage DNA directs the production of progeny phage by the host bacterium. The new phages burst from the host cell, killing it, and then infecting more bacteria (FIG. 3). There are many types of phages, many of which are capable of eradicating their bacterial hosts. Bacteriophages are among the most common and diverse entities in the biosphere. Bacteriophages occur abundantly in the biosphere, with different virions, genomes, and lifestyles. Phages only attack bacteria and have not been found to have diverse effects on humans or other animals. There are two types of phages: lytic or temperate. Only lytic phages are useful for developing therapeutic phage preparations. Lytic phages multiply inside the bacterial cell and release new phages, lysing and killing the host bacterial cell in the process (FIG. 3).

Phages were discovered to be antibacterial agents and were used in the former Soviet Republic of Georgia and the United States during 1920s and 1930s for treating bacterial infections. They had widespread use, including treatment of soldiers in the Red Army (1). However, they were abandoned for general use in the West for several reasons: Medical trials were carried out, but a basic lack of understanding of phages made these invalid; Antibiotics were discovered and marketed widely. They were easier to make, store and to prescribe; and Former Soviet research continued, but publications were mainly in Russian or Georgian languages and were unavailable internationally for many years. Their use has continued since the end of Cold War in Georgia and elsewhere in Central and Eastern Europe.

In recent years there has been renewed interest in phage therapy primarily because of the growing resistance of many strains of bacteria to existing antibiotics. The first regulated, randomized, double-blind clinical trial was reported in the Journal of Wounds Care in June 2009, which evaluated the safety and efficacy of a bacteriophage cocktail to treat infected venous leg ulcers in human patient (2). The FDA approved the study as a Phase I clinical trial. The study's results demonstrated the safety of therapeutic application of bacteriophages but did not show efficacy. Another controlled clinical trial in Western Europe (treatment of ear infections caused by Pseudomonas aeruginosa) was reported shortly in August 2009 (3). The study concludes that bacteriophage preparations were safe and effective for treatment of chronic ear infections in humans. Since 2006, the United States Food and Drug Administration (FDA) and United States Department of Agriculture (USDA) have approved several bacteriophage products. For example, the FDA approved LISTEX using bacteriophages on cheese to kill Listeria monocytogenes bacteria in 2006, giving them “generally recognized as safe (GRAS) status (4), and the same bacteriophage was approved for use on all food products in July 2007. Additionally, there have been numerous animal and other experimental clinical trials evaluating the efficacy of bacteriophages for various diseases, such as infected burns and wounds, and cystic fibrosis associated lung infections, among others (5). Phages are considered as being safe for therapeutic use, with few if any side effects having ever been reported. Pain was reported in one study, and was related to the rapid release of endotoxins as the phage lysed the bacteria, which is what happens also with antibiotic therapy.

In this post-antibiotic era, alternative therapies are eagerly sought by governments, public health agencies and the health care industry. Because bacteriophages can parasitize and kill specific bacteria; they are considered as biopharmaceuticals by US FDA for being non-parasitic to other microbes, plants or animals, or for being non-toxic and non-immunogenic to humans; and they have been approved by US FDA and EPA for food and environmental uses, where bacterial resistance to bacteriophage is minimal. Furthermore, there are no new safe and effective antibiotics on the horizon, and all efforts to develop a useful Staphylococcal vaccine have failed. Thus, bacteriophage treatment provides an alternative therapy for treatment of infections incited by Staphylococcus aureus.

### SUMMARY OF THE INVENTION

This invention describes a genetically modified Staphylococcus aureus bacteriophage VDX-10 comprising the DNA of the Staphylococcus aureus bacteriophage VDX-10 being altered by inserting a gene sequence that increases the ability of the bacteriophage to replicate faster as compared to unmodified bacteriophage VDX-10, wherein the gene sequence that increases the bacteriophage's ability to replicate faster can be the gene sequence that disables bacterial defenses against the bacteriophage.

In one aspect, the invention also provides a method of producing a genetically modified Staphylococcus aureus bacteriophage VDX-10 comprising inserting a gene sequence into the DNA of the Staphylococcus aureus bacteriophage VDX-10, wherein the gene sequence increases the ability of the bacteriophage to replicate faster as compared to unmodified bacteriophage VDX-10.

In another aspect, the invention also relates to a method of treating infection incited by Staphylococcal aureus in a patient, the method comprising administering an amount of a genetically modified Staphylococcal aureus bacteriophage VDX-10 to the patient effective to eliminating Staphylococcus bacteria cells, wherein the modified bacteriophage VDX-10 comprising the DNA of the bacteriophage VDX-10 being altered by inserting a gene sequence that increases the ability of the bacteriophage to replicate faster as compared to unmodified VDX-10 bacteriophage, wherein the gene sequence that increases the bacteriophage's ability to replicate faster can be the gene sequence that disables bacterial defenses against the bacteriophages,

wherein the infection can be respiratory infection such as ventilator-associated pneumonia (VAP), and wherein the VAP can be incited by methicillin-resistant Staphylococcal aureus (MRSA), methicillin-sensitive Staphylococcal aureus (MRSA), community-associated methicillin-resistant Staphylococcal aureus (CA-MRSA), or hospital-acquired methicillin-resistant Staphylococcal aureus (HA-MRSA);

alternatively, wherein the infection can be systemic infection such as bacteremia, and wherein the bacteremia can be incited by methicillin-resistant Staphylococcal aureus (MRSA) or methicillin-sensitive Staphylococcal aureus (MRSA).

In another aspect, the invention also relates to a method of treating ventilator-associated pneumonia (VIP) incited by Staphylococcal aureus in a patient, the method comprising administering an amount of a genetically modified VDX-10 bacteriophage to the patient effective to eliminating Staphylococcus bacteria cells, wherein the modified VDX-10 bacteriophage comprising the DNA of the bacteriophage being altered by inserting a gene sequence that increases the ability of the bacteriophage to replicate faster as compared to unmodified bacteriophage VDX-10, wherein the gene sequence that increases the bacteriophage's ability to replicate faster can be the gene sequence that disables bacterial defenses against the bacteriophages,

wherein the VAP is incited by methicillin-resistant Staphylococcal aureus (MRSA) or methicillin-sensitive Staphylococcal aureus (MSSA).

In another aspect, the invention also relates to a method of treating bacteremia incited by Staphylococcal aureus in a patient, the method comprising administering an amount of a genetically modified Staphylococcal aureus bacteriophage VDX-10 to the patient effective to eliminating Staphylococcus bacteria cells, wherein the modified bacteriophage VDX-10 comprising the DNA of the Staphylococcal aureus bacteriophage VDX-10 being altered by inserting a gene sequence that increases the ability of the bacteriophage to replicate faster as compared to unmodified bacteriophage VDX-10, wherein the gene sequence that increases the bacteriophage's ability to replicate faster can be the gene sequence that disables bacterial defenses against the bacteriophages,

wherein the bacteremia is incited by methicillin-resistant Staphylococcal aureus (MRSA) or methicillin-sensitive Staphylococcal aureus (MSSA).

### BRIEF DESCRIPTION OF THE DRAWINGS

FIG. 1 is a series of microscope images showing several bacteriophages, bacteriophage VDX-10 is on the right.

FIG. 2 is a set of images showing bacteriophage morphology.

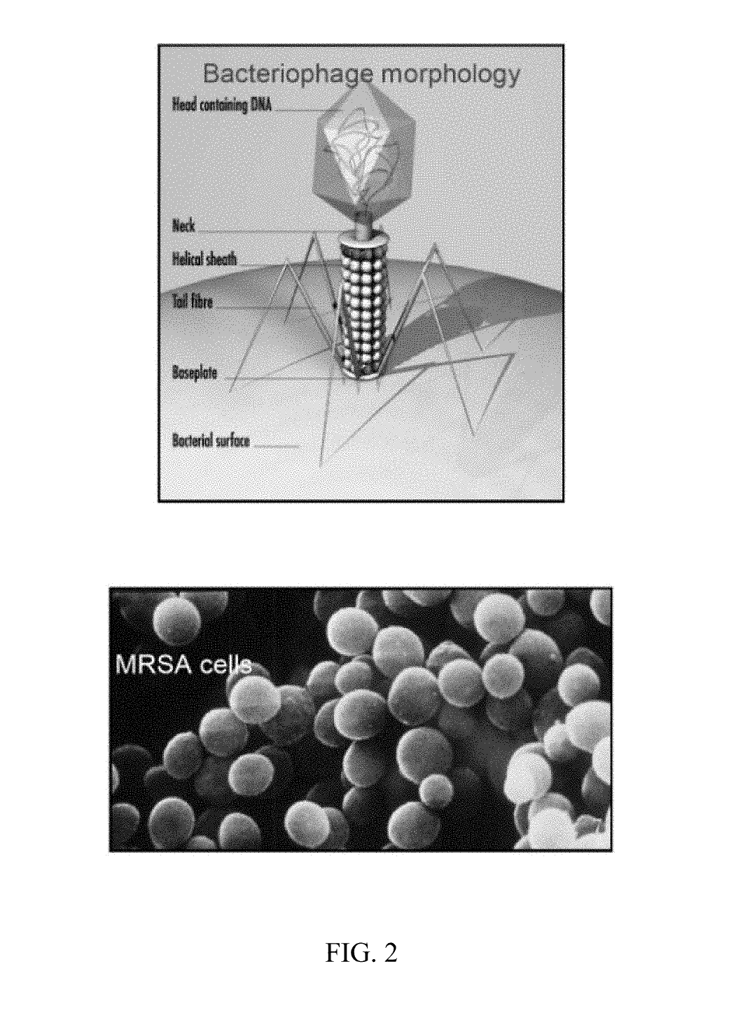

FIG. 3 is a set of illustrations detailing virus spread in the bacterium.

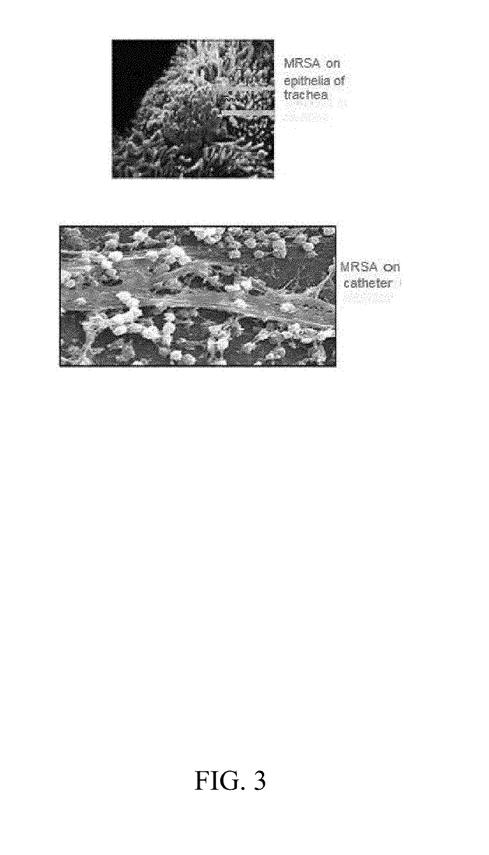

FIG. 4 is a set of microscope images showing MRSA, which are MRSA cells, MRSA on epithelia of trachea; and MRSA on catheter.

FIG. 5 shows the structure of T2 phage.

FIG. 6 is an electron microscope image VDX-10.

FIG. 7 is an electron microscope image VDX-10.

### DETAIL DESCRIPTIONS OF THE INVENTION

All illustrations of the drawings are for the purpose of describing selected versions of the present invention and are not intended to limit the scope of the present invention.

Many strains of common infectious bacteria including Staphylococcus aureus (S. aureus) and other Staphylococcal species are now resistant to most commercially-available antibiotics. The health threat and economic consequences of many common infections are now catastrophic in both the developing and the developed world. S. aureus is a common bacterium that may be found on the skin and in the nasal passages of health people. It is known to incite serious, life-threatening infections of the respiratory tract and cardiovascular system, including the blood stream, plus infections of bone, soft tissue and eye. Forms of S. aureus that are resistant to methicillin and other antibiotics are known as Methicillin-Resistant Staphylococcal aureus (MRSA). Vancomycin is generally used to treat resistant Staphylococcal infections with some success. However, a more resistant form of S. aureus, known as Vancomycin-Intermediate-Resistant S. aureus (VISA) has emerged to complicate treatment. The most troublesome form of antibiotic-resistant S. aureus is known as Vancomycin-Resistant S. aureus (VRSA).

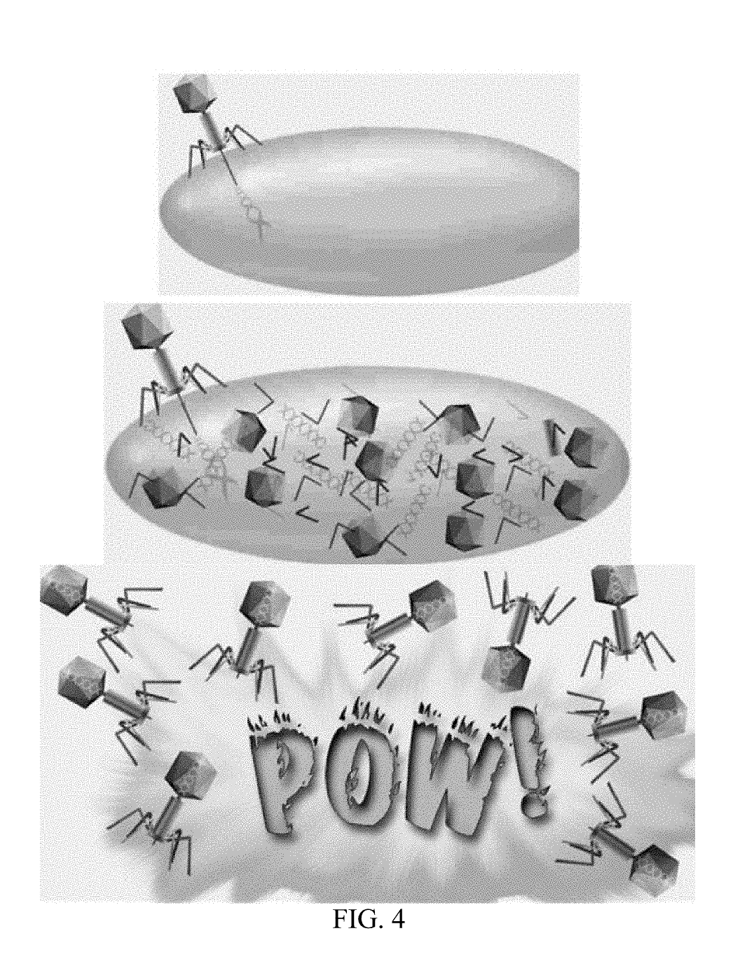

MRSA (FIG. 4) is an antibiotic-resistant form of S. aureus that is responsible for infections in patients of all ages. Infection by MRSA is typically associated with particular settings such as hospitals and long-term care facilities, and is common among patient groups that have prolonged hospitalizations, past antimicrobial use, indwelling catheters, decubitis ulcers and postoperative surgical wounds, as well as those who require intravenous drugs or treatment with enteral feedings or dialysis. Infections incited by MRSA present a considerable dilemma to clinician, since therapeutic options are limited and suboptimal dosing contributes to greater resistance, heightened mortality and increased length of hospital stay.

Methicillin-Sensitive Staphylococcus aureus (MSSA) is a form of S. aureus that is or may be resistant to many common antibiotics, but that is sensitive to methicillin. MSSA is also responsible for serious infections in patients of all ages. Infection by MSSA may be encountered in hospital, clinical or community settings, and is increasingly common among patients groups that have had recent hospitalizations or otherwise crowded conditions, such as among sporting teams, military situations, confinement or incarceration, or, who were in close contact with animals.

Ventilator-Associated Pneumonia (VAP) is characterized as pneumonia of infectious origin in a patient on a ventilator, which results in fluid accumulation in lung alveoli. VAP is distinguished from other pneumonias by the inciting pathogen, the antibiotic treatments administered, and the methods of diagnosis, prognosis and prevention. In order to have VAP, the patient must be on a ventilator within the past 48 hours.

MRSA-Incited Ventilator-Associated Pneumonia (MRSA-VAP) means VAP that is incited by MRSA, one of the key pathogens associated with VAP. Patients on ventilator are already sick and likely to become infected with MRSA and develop MRSA-VAP, which has a high rate of mortality among all patients on ventilators, and most especially among the elderly. MSSA-Incited Ventilator-Associate Pneumonia (MSSA-VAP) means VAP that is incited by MSSA.

Bacteremia is the presence of bacteria in the blood, which may be caused by dental work, catheterization of urinary tract, surgical treatment of an abscess of infected wound, or colonization of indwelling devices, especially intravenous and intra-cardiac catheters, urethral catheters or ostomy devices and tubes. Bacteremia secondary to infection usually originates in the genitourinary (GU) or gastrointestinal (GI) tract, or on the skin. Chronically ill patients, immunocompromised patients and injection drug users have an increased risk of bacteremia. MRSA is a common, dangerous and difficult-to-treat inciting agent of bacteremia. In other words, MRSA-Bacteremia is MRSA-incited bacteremia; and MSSA-Bacteremia is MSSA-incited bacteremia.

Here we report production of a genetically modified and enhanced Staphylococcus aureus bacteriophage VDX-10 and its use in the treatment Ventilated Associated Pneumonia (VAP) or bacteremia as incited by MRSA or MSSA.

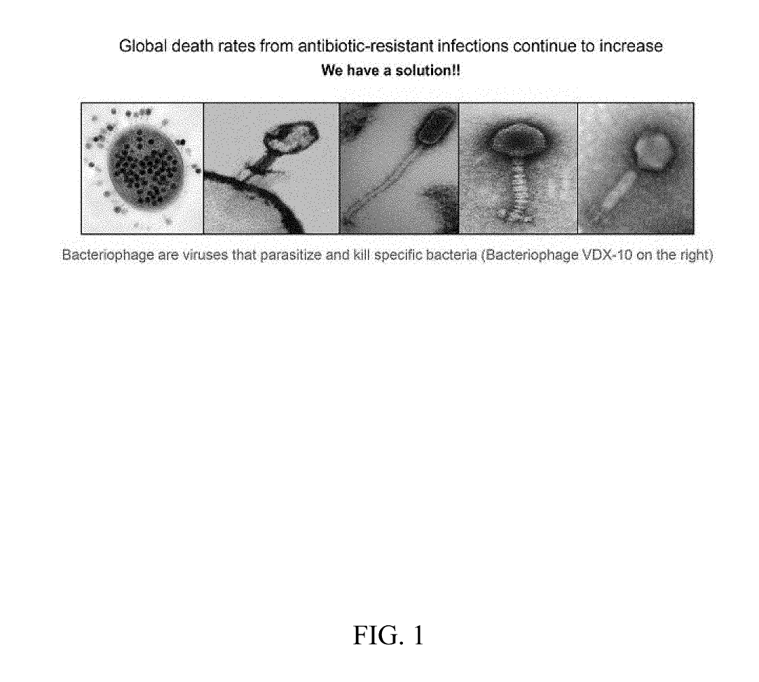

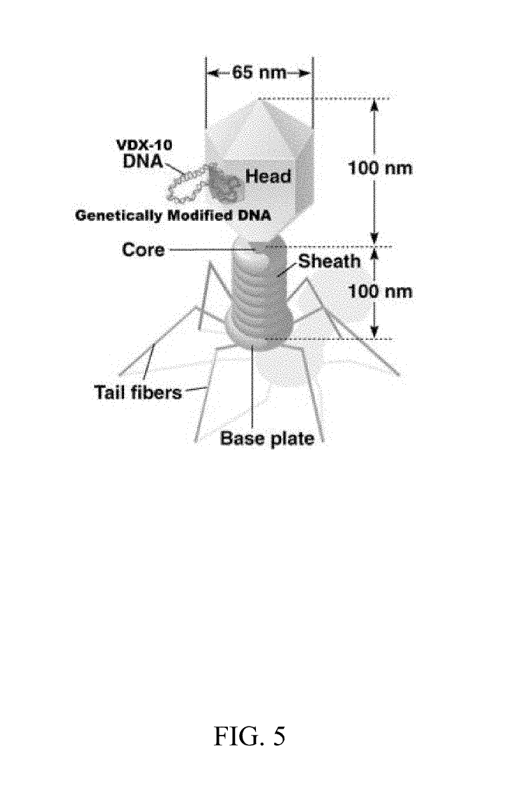

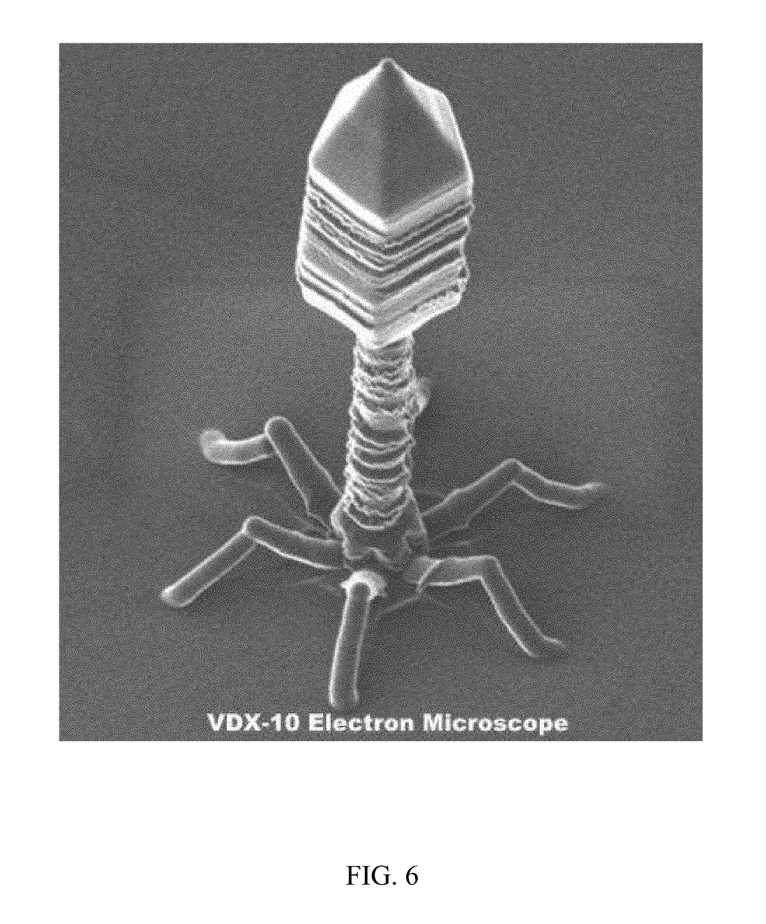

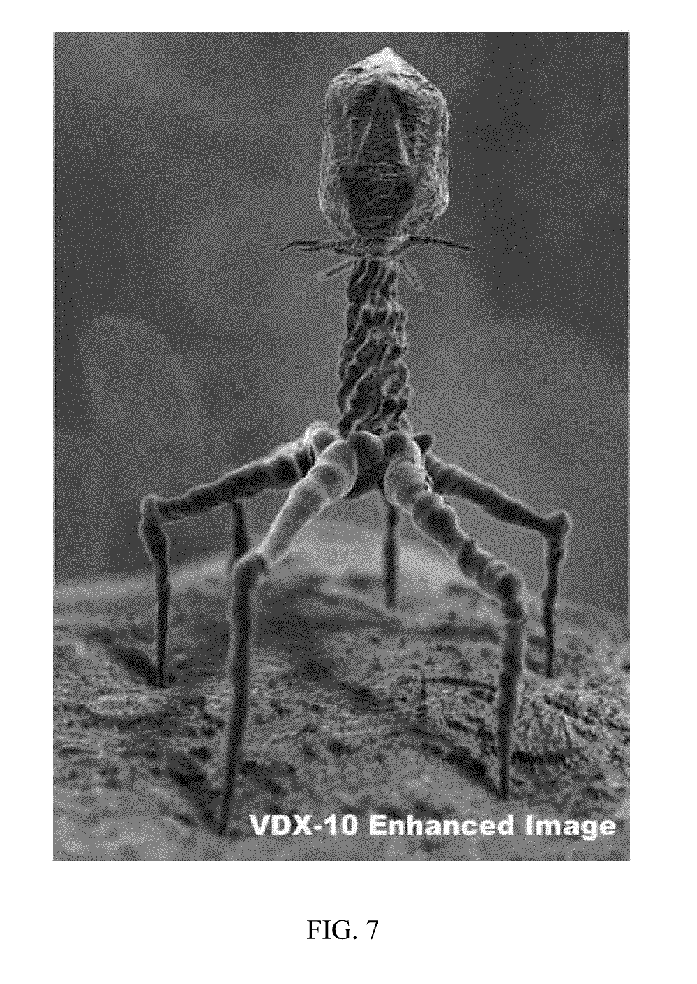

Using natural selection and isolation after numerous generational cycles, the best suited phages such as VDX-10 phage (FIGS. 5, 6 and 7) are chosen as a candidate for gene modification. These naturally superior phages are then genetically altered to increase their ability to reproduce faster. The genetic sequence that controls their production is changed by using the CRISPR process tool to remove the gene sequence from the best performing natural phage to other phages in the control group which increase their ability to produce faster. These cDNA altered phages are then fermented into colonies and the modified phages can be used as a treatment for Staphylococcal bacterial infection. Faster reproduction increases the phage population more rapidly allowing the phage community to combat the Staphylococcus bacteria population more aggressively. By adding additional genetic material to the natural high performance phage results in the production a new organism that does not occur in nature. For example, the natural Staphylococcus aureus phages (e.g., VDX-10 bacteriophages, the right image of FIG. 1) are modified by inserting a unique gene that disables bacterial defenses against unique cDNA bacteriophage using CRISPR/CAS technology and lambda Red technology, which would broaden the host range within the targeted species.

Staphylococcus aureus bacteriophage VDX-10 (FIGS. 5, 6 and 7) has been identified to target Staphylococcus aureus (MSSA & MRSA). A genetically modified and enhanced VDX-10 is the invention hereafter described. The DNA of the VDX-10 has been altered by inserting a gene sequence that increases the bacteriophage's ability to replicate faster, thereby increasing its numbers and increasing its effectiveness in eliminating Staphylococcus bacteria cells. The invention is a unique genetically modified phage that can be programmed to target various harmful life threatening bacteria. The unmodified VDX-10 bacteriophages and modified bacteriophages are produced in batches or fed-batches in bioreactor and can be amplified in S. aureus strains according to the method described previously (6).

Many applications in bacteriophage research require pure and highly concentrated phage suspensions. For the use in phage therapy, purification steps are needed, depending on the type of applications, such as medical, agricultural or veterinary application. The purification process of modified bacteriophage VDX-10 is designed to reach the optimal purity required by pharmacopeia for the proposed route of administration. A process of tangential flow filtration, chromatography or density gradient centrifugation or a combination thereof would be used. For example, Staphylococcus aureus phage VDX-10 from bacterial lysate was purified using methacrylate monoliths (Convective Interaction Media® [CIM] monolithic columns) (7, 8). With a single step purification method, more than 99% of host cell DNA and more than 90% of proteins were removed, with 60% recovery of viable phages. Comparable results were obtained when the purification method was scaled up from a CIM monolithic disk to a larger CIM monolithic column. Protein content and endotoxin content at different stages of phage purification are determined according to the methods described previously (9).

Many Staphylococcus aureus isolates comprising MRSA and MSSA strains are collected and used to assess the bactericidal activity of two Staphylococcal phages, VDX-10 and genetically modified VDX-10, respectively. Both unmodified VDX-10 and genetically modified VDX-10 are assessed on a panel of Staphylococcus aureus isolates including both MSSA and MRSA according to the procedure previously (9). The efficacy of genetically modified VDX-10 bacteriophage in vivo can be evaluated using an animal model as described previously (9). Both unmodified VDX-10 and genetically modified bacteriophage VDX-10 product can be administered to a patient in the treatment of infections such as VAP or bacteremia incited by MRSA or MSSA, wherein the phage can be administered orally, topically, or systemically, and wherein the patient can be an animal or a human.

As used herein, “a” or “an” means one or more (or at least one), for example, a gene sequence means at least one gene sequence.

Although the invention has been explained in relation to its preferred embodiment, it is to be understood that many other possible modifications and variations can be made without departing from the spirit and scope of the invention.

### REFERENCES

1. Sulakvelldze A; Alavidze Z; and Morris Jr., J. G. (March 2001) “Bacteriophage Therapy.” Antimicrobial Agents & Chemotherapy 45(3):649-659.
2. Rhoads, D D; Wolcott, R D; Kuskowski, M A; Wolcott, B M; Ward, L S; and Sulakvelidze, A (June 2009) “Bacteriophage Therapy of Venous Leg Ulcers in Humans: Results of Phase I Safety Trial.” Journal of Wound Care 18(6): 237-238, 240-243.
3. Wright, A; Hawkins, C H; Anggard, E E; and Harper, D R (August 2009) “A Controlled Clinical Trial of a Therapeutic Bacteriophage Preparation in Chronic Otitis Due to Antibiotic-Resistant Pseudomonas aeruginosa: a Preliminary Report of Efficacy.” Clinical Otolaryngology 34 (4): 349-357.
4. U.S. FDA/CFSAN: Agency Response Letter, GRAS Notice No. 000198.
5. Lu, T K; and Collins, J J (2007) “Dispersing Biofilms with Engineered Enzymatic Bacteriophage.” Proceedings of National Academy of Sciences USA 104 (27):11197-11202.
6. Nirmal, K G P; Sudarson, S; Paul, V D; Nandini, S; Sanjeev, R.; Hariharan, S; Spiram, B; and Padmanabhan, S (2012) “Use of Prophage Free Host for Achieving Homogenous Population of Bacteriophages: New Findings.” Virus Research 169 (1), 182-187.
7. Kramberger P; Honour, R. C.; Herman, R E; Smrekar, F.; and Peterka, M (2010) “Purification of the Staphylococcus aureus Bacteriophages VDX-10 on Methacrylate Monoliths.” J. Virol. Methods 166(1-2), 60-64.
8. Adriaenssens, E M; Lehman, S M; Vandersteegen K; Vandenheuvel D; Philippe, D L; Cornelissen, A; Clokie M R J; Garria A J; De Profit, M; Maes, M; and Lavigne R (2012) “CIM® Monolithic Anion-Exchange Chromatography As a Useful Alternative to CsCl Gradient Purification of Bacteriophage Particles.” Virology 434(2):265-270.
9. Narasimhaiah, M H; Asrani, J Y; Palaniswamy S M; Bhat, J; George S E; Srinivasan, R; Vipra, A; Desai, S N; Junjappa, R P; Roy, P; Sriram, B; and Padmanabhan, S (2013) “Therapeutic Potential of Staphylococcal Bacteriophages for Nasal Decolonization of Staphylococcus aureus in Mice.” Advances in Microbiology 3, 52-60.

What is claimed is:

1. A genetically modified Staphylococcus aureus bacteriophage VDX-10 comprising the DNA of the bacteriophage VDX-10 being altered by inserting a gene sequence that increases the ability of the bacteriophage to replicate faster as compared to unmodified Staphylococcus aureus bacteriophage VDX-10.

2. The genetically modified bacteriophage VDX-10 according to claim 1, wherein the gene sequence that increases the bacteriophage's ability to replicate faster is the gene sequence that disables bacterial defenses against the bacteriophage.

3. A method of producing a genetically modified Staphylococcus aureus bacteriophage VDX-10, the method comprising inserting a gene sequence into the DNA sequence of the Staphylococcus aureus bacteriophage VDX-10, wherein the gene sequence increases the ability of the bacteriophage to replicate faster as compared to unmodified bacteriophage VDX-10.

4. A method of treating infection incited by Staphylococcal aureus in a patient, the method comprising administering an amount of a genetically modified Staphylococcus aureus bacteriophage VDX-10 to the patient effective to eliminating Staphylococcus bacteria cells, wherein the modified bacteriophage VDX-10 comprising the DNA of the Staphylococcus aureus bacteriophage VDX-10 being altered by inserting a gene sequence that increases the ability of the bacteriophage to replicate faster as compared to unmodified bacteriophage VDX-10.

5. The method according to claim 4, wherein the gene sequence that increases the bacteriophage's ability to replicate faster is the gene sequence that disables bacterial defenses against the bacteriophage.

6. The method according to claim 4, wherein the infection is respiratory infection.

7. The method according claim 6, wherein the respiratory infection is ventilator-associated pneumonia (VAP).

8. The method according claim 7, wherein the VAP is incited by methicillin-resistant Staphylococcal aureus (MRSA).

9. The method according claim 7, wherein the VAP is incited by methicillin-sensitive Staphylococcal aureus (MSSA).

10. The method according claim 7, wherein the VAP is incited by community-associated methicillin-resistant Staphylococcal aureus (CA-MRSA).

11. The method according claim 7, wherein the VAP is incited by hospital-acquired methicillin-resistant Staphylococcal aureus (HA-MRSA).

12. The method according to claim 4, wherein the infection is systemic infection.

13. The method according claim 12, wherein the systemic infection is bacteremia.

14. The method according claim 13, wherein the bacteremia is incited by methicillin-resistant Staphylococcal aureus (MRSA).

15. The method according claim 13, wherein the bacteremia is incited by methicillin-sensitive Staphylococcal aureus (MSSA).

16. A method of treating ventilator-associated pneumonia (VIP) incited by Staphylococcal aureus in a patient, the method comprising administering an amount of a genetically modified Staphylococcus aureus bacteriophage VDX-10 to the patient effective to eliminating Staphylococcus bacteria cells, wherein the modified bacteriophage VDX-10 comprising the DNA of the Staphylococcus aureus bacteriophage VDX-10 being altered by inserting a gene sequence that increases the ability of the bacteriophage to replicate faster as compared to unmodified bacteriophage VDX-10.

17. The method according claim 16, wherein the VAP is incited by methicillin-resistant Staphylococcal aureus (MRSA).

18. The method according claim 16, wherein the VAP is incited by methicillin-sensitive Staphylococcal aureus (MSSA).

19. A method of treating bacteremia incited by Staphylococcal aureus in a patient, the method comprising administering an amount of a genetically modified Staphylococcal aureus bacteriophage VDX-10 to the patient effective to eliminating Staphylococcus bacteria cells, wherein the modified bacteriophage VDX-10 comprising the DNA of the Staphylococcal aureus bacteriophage VDX-10 being altered by inserting a gene sequence that increases the ability of the bacteriophage to replicate faster as compared to unmodified bacteriophage VDX-10.

20. The method according claim 19, wherein the bacteremia is incited by methicillin-resistant Staphylococcal aureus (MRSA) or methicillin-sensitive Staphylococcal aureus (MSSA).
